(19) United States
(12) Patent Application Publication
(10) Pub. No.: US 2016/0051252 A1
(43) Pub. Date: Feb. 25, 2016

(54) ANCHORS AND CINCHING FOR TISSUE OPPOSITION

(71) Applicant: Boston Scientific Scimed, Inc., MAPLE GROVE, MN (US)

(72) Inventors: Paul SMITH, Smithfield, RI (US); Gary S. KAPPEL, Acton, MA (US); Samuel RAYBIN, Marlborough, MA (US); John B. GOLDEN, Norton, MA (US); Liam ROLLE, Boston, MA (US); Jason WEINER, Grafton, MA (US); Paul MANNION, Shrewsbury, MA (US); Thomas JONES, Franklin, MA (US); Michael K. FORD, Waltham, MA (US); Kirsten VIERING, Watertown, MA (US); Kerry L. GRANT, Northbridge, MA (US); Naroun SUON, Lawrence, MA (US); Michael POWERS, Pepperell, MA (US)

(21) Appl. No.: 14/830,349

(22) Filed: Aug. 19, 2015

Related U.S. Application Data
(60) Provisional application No. 62/040,276, filed on Aug. 21, 2014.

(51) Int. Cl. A61B 17/04; A61B 17/10; A61B 17/08

(52) U.S. Cl. CPC A61B 17/0466; A61B 17/083; A61B 17/105; A61B 17/0401; A61B 2017/0409; A61B 2017/0414; A61B 2017/0417; A61B 2017/0419; A61B 2017/0464

(57) ABSTRACT

A system for treating tissue includes a first anchoring device including a first anchoring element at a distal end thereof for anchoring the first anchoring device in a first target portion of tissue extending about a periphery of a tissue defect to be treated and a first suture receiving feature at a proximal end thereof. The system also includes a second anchoring device including a second anchoring element at a distal end thereof for anchoring the second anchoring device in a second target portion of tissue about the periphery of the tissue defect and a second suture receiving feature at a proximal end thereof. Furthermore, the system includes a suture configured to be passed through the first and second suture receiving features such that a tension applied to the suture draws the first and second anchoring devices toward one another to close the tissue defect.

### PRIORITY CLAIM

The present disclosure claims priority to U.S. Provisional Patent Application Ser. No. 62/040,276 filed, Aug. 21, 2014; the disclosure of which is incorporated herewith by reference.

### BACKGROUND

Physicians have been increasingly willing to perform more aggressive interventional and therapeutic endoscopic procedures including, for example, the removal of larger lesions (e.g., cancerous masses), tunneling under the mucosal layer of the gastrointestinal (GI) tract to treat tissue below the mucosa, full thickness removal of tissue, the treatment of issues on other organs by penetrating and passing of the GI tract, and the endoscopic treatment/repair of post-surgical issues (e.g., post-surgical leaks, breakdown of surgical staple lines, anastomic leaks). These procedures my increase the risk of perforating the wall of the GI tract, or may require closure of the GI tract wall as part of the procedure. Endoscopic closure can save costs for the hospital and provide benefits to the patient. However, current devices for tissue close are often difficult to use and time consuming. In addition, current devices may be insufficient to close for certain perforation or to treat certain condition and anatomies such as, for example, large wounds created in the GI tract.

### SUMMARY

The present disclosure is directed to a system for treating tissue, comprising a first anchoring device including a first anchoring element at a distal end thereof for anchoring the first anchoring device in a first target portion of tissue extending about a periphery of a tissue defect to be treated and a first suture receiving feature at a proximal end thereof, a second anchoring device including a second anchoring element at a distal end thereof for anchoring the second anchoring device in a second target portion of tissue about the periphery of the tissue defect and a second suture receiving feature at a proximal end thereof, and a suture configured to be passed through the first and second suture receiving features such that a tension applied to the suture draws the first and second anchoring devices toward one another to close the tissue defect.

In an embodiment, the system may further comprise a third anchoring device including a third anchoring element at a distal end thereof for anchoring the third anchoring device in a third target portion of tissue about the periphery of the tissue defect and a third suture receiving feature at a proximal end thereof.

In an embodiment, each of the first and second suture receiving features may be a hook curving away from the proximal end of the first and second anchoring devices toward the distal ends thereof.

In an embodiment, each of the first and second anchoring elements may be one of a barb, a projection, a threading and a T-tag.

In an embodiment, the system may further comprise a delivery tool including a longitudinal member extending longitudinally from a proximal end to a distal end and including a lumen extending therethrough, the lumen sized and shaped to movably receive the first and second anchoring devices longitudinally therein.

In an embodiment, the delivery tool may further include a distal jaw coupled to the longitudinal member such that the distal jaw is longitudinally movable relative thereto, a space between the distal end of the longitudinal member and the distal jaw configured to receive the target tissue therebetween so that one of the first and second anchoring devices may be passed therethrough.

In an embodiment, the distal jaw may include a slot extending longitudinally therethrough an open to a lateral edge thereof.

In an embodiment, each of the first and second receiving features may include a tortuous path.

In an embodiment, the tortuous path may be formed of a hole extending through a proximal portion of each of the first and second anchoring devices and a pair of hooks extending proximally from the proximal portion, wherein a space extending through each of the pair of hooks is offset from one another.

In an embodiment, the tortuous path may be formed of a first and second hole extending laterally through a portion of the first and second anchoring devices, the first and second holes in communication with one another.

The present disclosure is also directed to a system for treating a tissue defect, comprising a delivery tube extending from a proximal end to a distal end and including a lumen extending therethrough, a first clip movably housed within the lumen, the first clip including a first pair of arms at a distal end thereof, the pair of arms movable between an open configuration, in which the first pair of arms are separated from one another to receive a first portion of target tissue therebetween, and a closed configuration, in which the first pair of arms are drawn toward one another to grip tissue therebetween, the first clip biased toward the closed configuration so that when the first clip is received within the lumen of the delivery tube, an interior surface of the lumen constrains a proximal portion of the first clip to hold the first clip in the open configuration, and a second clip movably housed within the lumen proximally of the first clip, the second clip including a second pair of arms at a distal end thereof, the second pair of arms movable between an open configuration and a closed configuration, the second clip biased toward the closed configuration so that when the second clip is received within the lumen of the delivery tube, an interior surface of the lumen constrains a proximal portion of the second clip to hold the second clip in the open configuration.

In an embodiment, the system may further comprise a suture connected to the first clip and extending through the proximal portion of the second clip such that, when the first and second clip have been applied to target portions along a periphery of a tissue defect, tensioning of the suture draws the first and second clip toward one another.

In an embodiment, the system may further comprise an elastic band configured to be gripped by the first and second pairs or arms.

In an embodiment, the suture may include ratcheting features along a portion of a length thereof, the ratcheting features permitting the suture to be drawn proximally through the proximal portion of the second clip while preventing the suture from sliding distally therethrough.

In an embodiment, the system may further comprise a mandrel for moving the first and second clips distally relative to the delivery tube.

The present disclosure also relates to a method for treating a tissue defect, comprising placing a first anchoring device in a first target portion of tissue about a periphery of a tissue defect to be treated, the first gripping device including a first suture receiving feature at a proximal end thereof, placing a second anchoring device in a second target portion of tissue about the periphery of the tissue defect, the second anchoring device including a second suture receiving feature at a proximal end thereof, passing a suture through the first and second suture receiving features, and tensioning the suture to draw the first and second anchoring devices toward one another.

### BRIEF DESCRIPTION

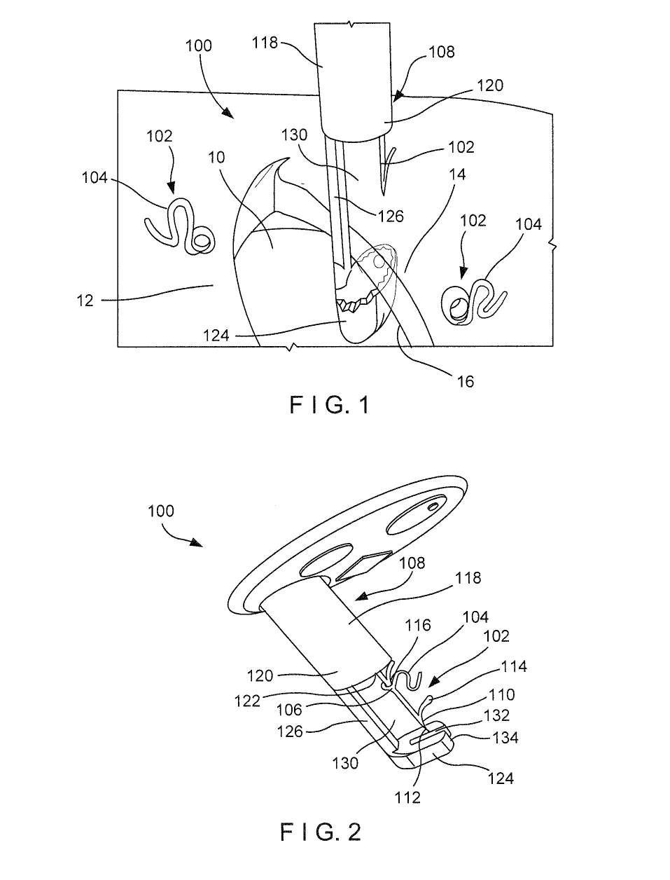

FIG. 1 shows a perspective view of a system according to a first exemplary embodiment of the present disclosure;

FIG. 2 shows another perspective view of the system of FIG. 1;

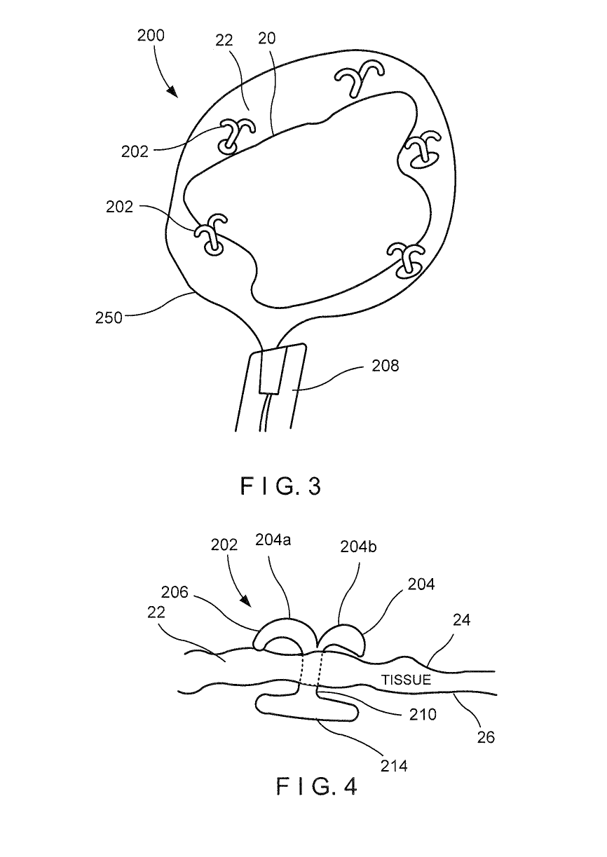

FIG. 3 shows a perspective view of a system according to a second exemplary embodiment of the present disclosure;

FIG. 4 shows a side view of an anchor of the system of FIG. 3;

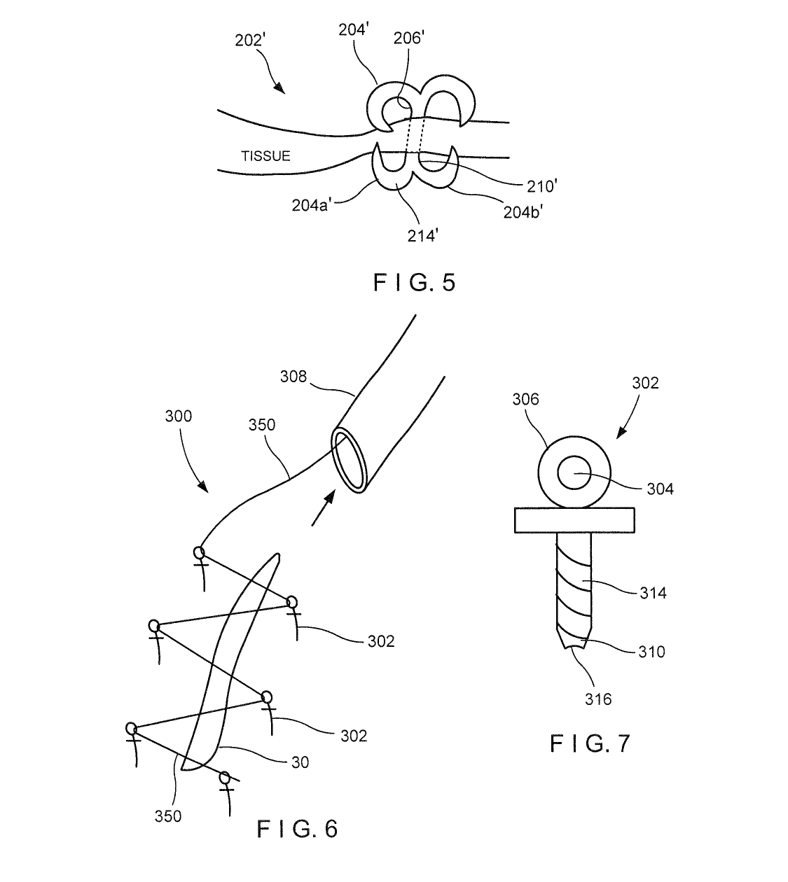

FIG. 5 shows a side view of an anchor according to an alternate embodiment;

FIG. 6 shows a perspective view of a system according to a third exemplary embodiment of the present disclosure;

FIG. 7 shows a side view of an anchor of the system if FIG. 6;

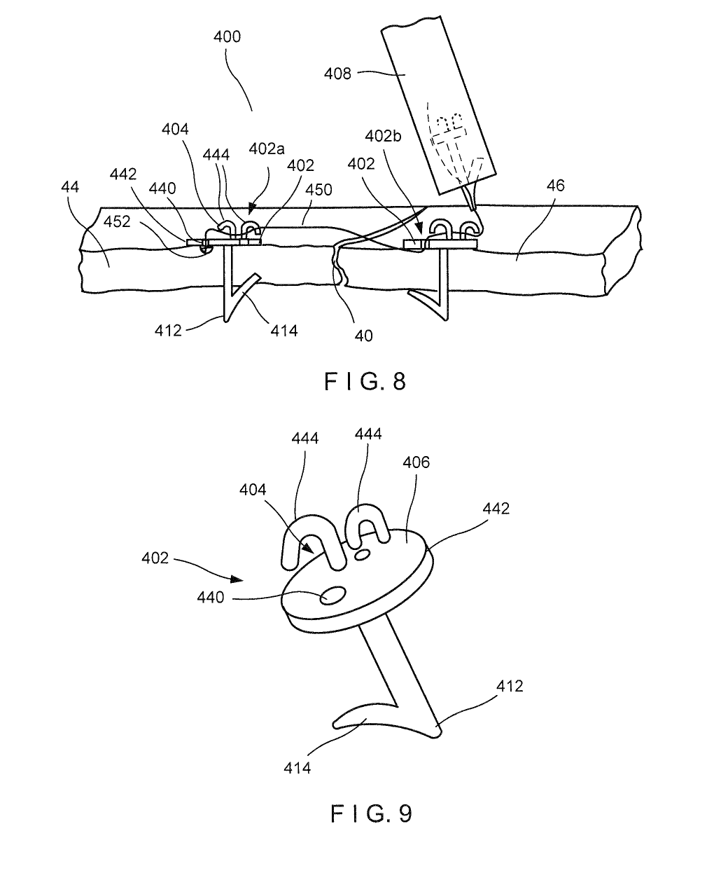

FIG. 8 shows a side view of a system according to a fourth exemplary embodiment of the present disclosure;

FIG. 9 shows a perspective view of an anchor of the system of FIG. 8;

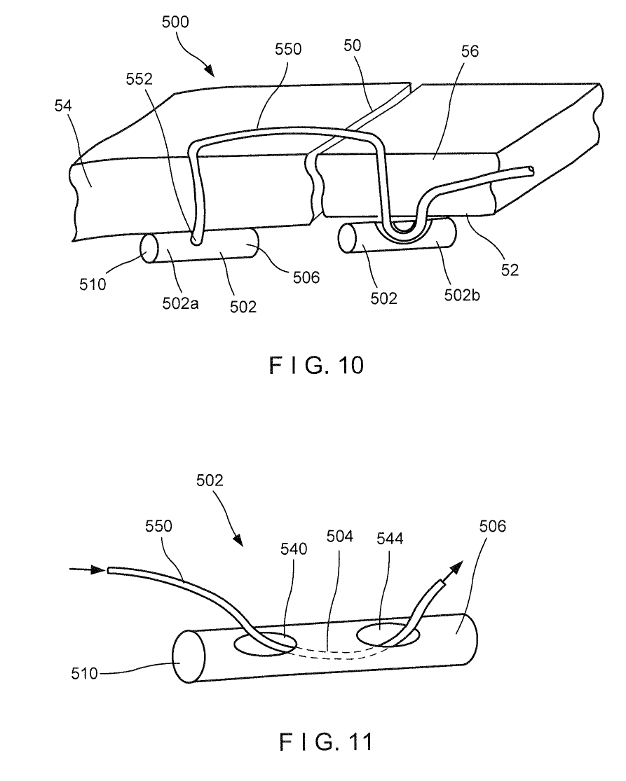

FIG. 10 shows a side view of a system according to a fifth exemplary embodiment of the present disclosure;

FIG. 11 shows a perspective view of an anchor of the system of FIG. 10;

FIG. 12 shows a perspective view of a system according to a sixth exemplary embodiment of the present disclosure, in a first configuration;

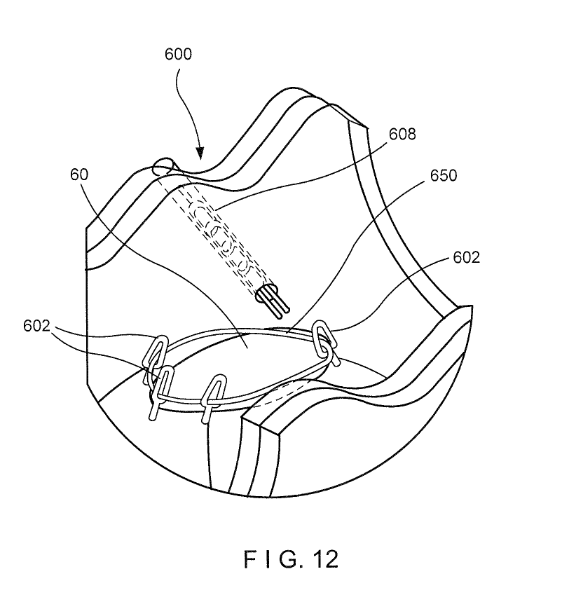

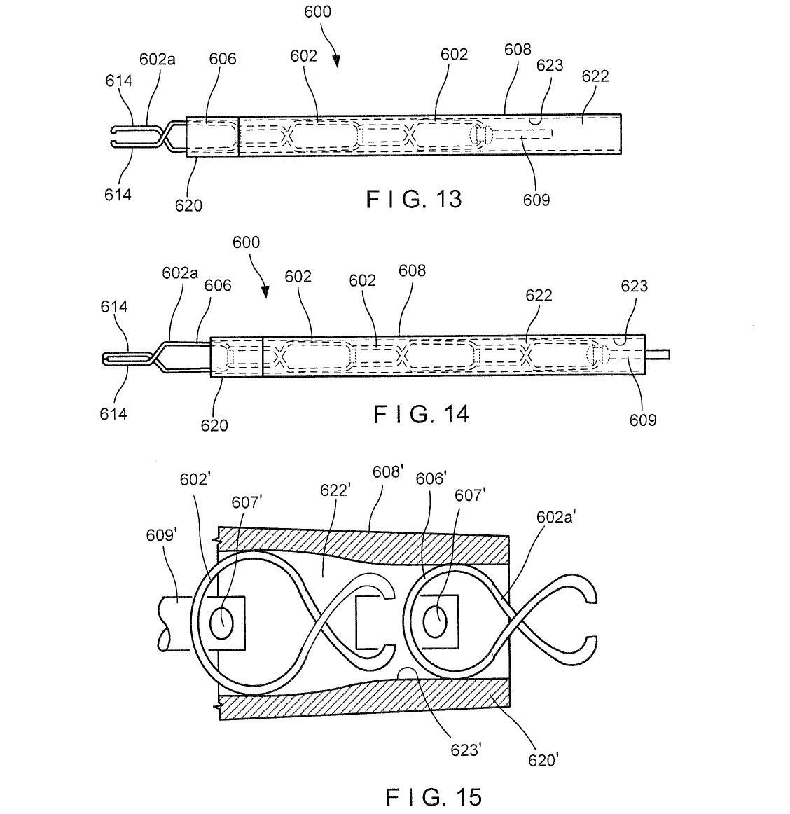

FIG. 13 shows a side view of the system of FIG. 12, in a first configuration;

FIG. 14 shows a side view of the system of FIG. 12, in a second configuration;

FIG. 15 shows a cross-sectional side view of a system according to an alternate embodiment of the present disclosure;

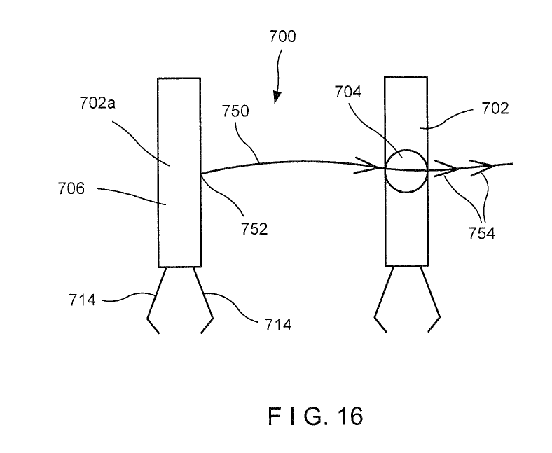

FIG. 16 shows a side view of a system according to a seventh exemplary embodiment of the present disclosure;

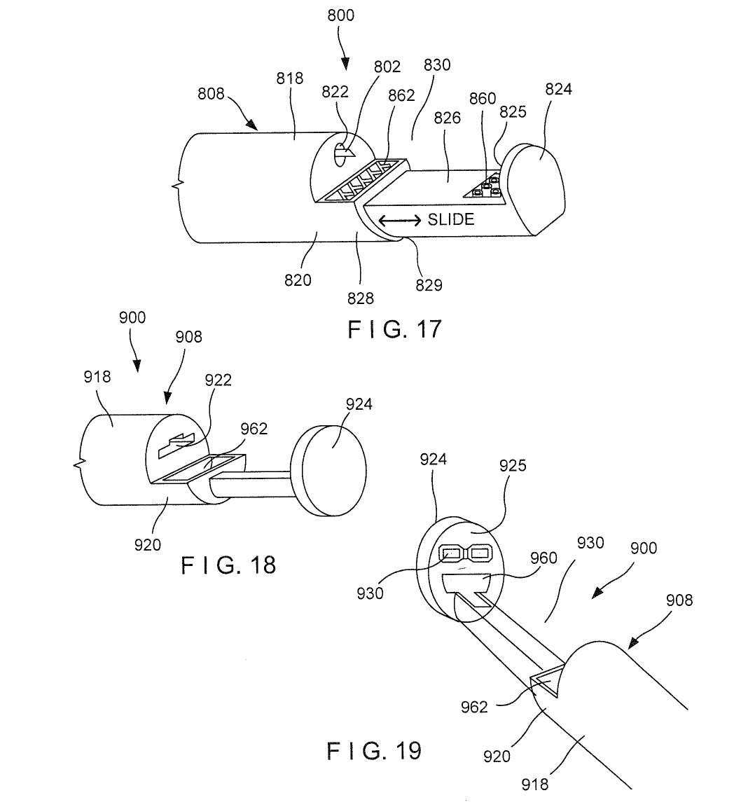

FIG. 17 shows a perspective view of a system according to an eighth exemplary embodiment of the present disclosure;

FIG. 18 shows a perspective view of a delivery tool according to another exemplary embodiment of the present disclosure;

FIG. 19 shows another perspective view of the delivery tool of FIG. 18;

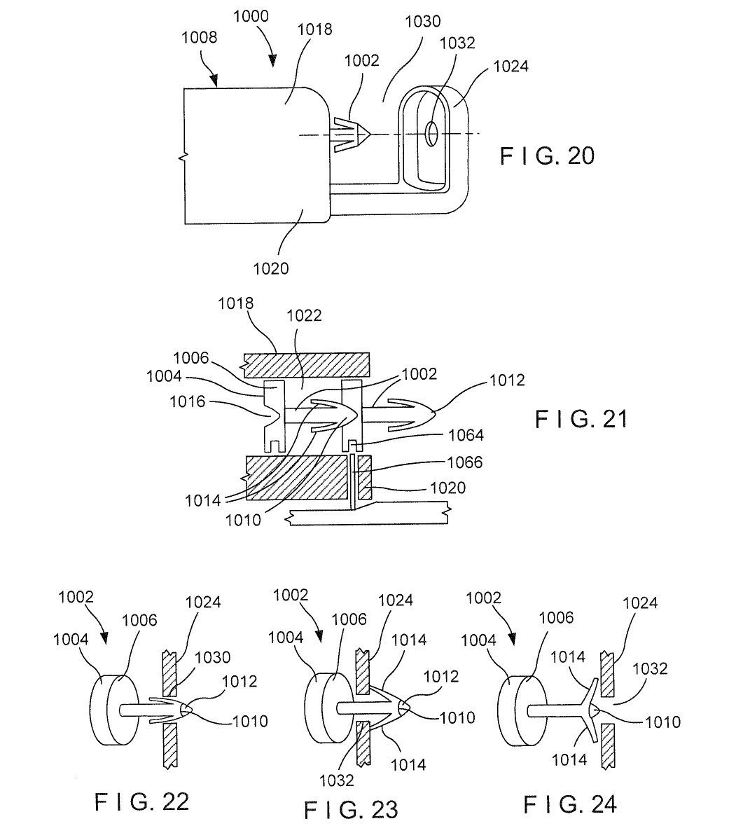

FIG. 20 shows a perspective view of a system according to another exemplary embodiment of the present disclosure;

FIG. 21 shows a cross-sectional view of a portion of the system of FIG. 20;

FIG. 22 shows a perspective view of a tissue joining device of the system of FIG. 20, in a first configuration;

FIG. 23 shows a perspective view of the tissue joining device of FIG. 22, in a second configuration; and

FIG. 24 shows a perspective view of the tissue joining device of FIG. 22, in a third configuration.

### DETAILED DESCRIPTION

The present disclosure may be further understood with reference to the following description and the appended drawings, wherein like elements are referred to with the same reference numerals. The present disclosure is directed to devices for the treatment of tissue and, in particular endoscopic tissue treatment devices. Exemplary embodiments of the present disclosure describe anchors positioned about a periphery of a tissue opening and drawn toward one another via sutures, loops or elastic bands to close the tissue opening. It should be noted that the terms “proximal” and “distal” are intended to refer to a direction toward (proximal) and away from (distal) a user of the device.

As shown in FIGS. 1 and 2, a system 100 according to a first exemplary embodiment of the present disclosure comprises a plurality of anchors 102, each anchor 102 configured to be positioned in tissue along a periphery of a tissue opening 10 and including a hook 104 at a proximal end 106 thereof for receiving a portion of a suture, loop or elastic band therein such that upon insertion of a desired number of anchors 102 about a periphery of the tissue opening 10, a suture may be passed through the hook 104 of each of the anchors 102. The suture may then be tightened to draw the anchors 102 toward one another, drawings edges of the tissue opening 10 toward one another to close the tissue opening. The anchors 102 may be delivered to a target tissue 12 about the tissue opening 10 via a delivery tool 108 sized and shaped to be inserted through, for example, a working channel of an endoscope.

Each anchor 102 extends from the proximal end 106 to a distal end 110. The distal end 110 includes a sharpened distal tip 112 for piercing a target tissue into which the anchor 102 is to be inserted. The distal end 110 may also include, for example, a barb or projection 114, which prevents the distal end 110 from disengaging tissue into which it has been inserted. The projection 114 may be biased toward a laterally outward position, as shown in FIG. 1. The projection 114, however, may be constrained by the delivery tool 108 to remain in an insertion configuration, in which it is moved toward a longitudinal axis of the anchor 102, as will be described in further detail below. In the insertion configuration, the projection 114 passes more easily into target tissue 12 via the sharpened distal tip 112. Once the anchor 102 is inserted distally through the target tissue 12, the projection 114 extends distally from a distal surface 16 thereof and reverts to the biased laterally outward position to prevent the anchor 102 from being drawn proximally therefrom.

The proximal end 106 of each of the anchors 102 includes the hook 104 extending laterally therefrom so that, when hook extends from a proximal surface 14 of tissue 12 in which the anchor 102 is embedded. The hook 104 is shaped so that, once a desired number of anchors 102 has been placed about a periphery of the tissue opening 10, a suture may be passed and received through each of the hooks 104 of the anchors 102. Once the suture has been received within the hooks 104, the suture may be tightened to draw edges of the tissue opening 10 toward one another, closing the tissue opening 10. The hook 104 also aids in holding the anchor 102 in place, within the target tissue 12. The hook 104 may be biased toward the hook shape but may be deformable to be received within a lumen 122 of the deliver tool 108. The proximal end 106 may also include, for example, a recess 116 sized and shaped to receive the distal end 110 of a subsequent anchor 102 received within the lumen 122 of the delivery tool 108. Although the exemplary embodiments show and describe a substantially hook-shaped element 104, the anchors 102 may include any of a variety of different suture-receiving features at the proximal end 106.

The delivery tool 108 comprises a longitudinal member 118 and a distal jaw 124 longitudinally movable relative thereto such that the target tissue 12 may be received in a space 130 between a distal end 120 of the longitudinal member 118 and the distal jaw 124. The longitudinal member 118 extends longitudinally from a proximal end (not shown) to the distal end 120 and includes a lumen 122 extending therethrough. The lumen 122 is sized and shaped to receive the anchors 102 longitudinally therein. The anchors 102 may be received therein with the proximal end 106 of the distal-most anchor 102 in contact with the distal end 110 of an immediately proximal anchor 102. In particular, the distal tip 112 of the immediately proximal anchor 102 may be received within the recess 116 at the proximal end 106 of the distal-most anchor 102. The anchors 102 may be moved distally through the lumen 122 via, for example, a mandrel slidably received in the lumen 122 in contact with a proximal-most one of the anchors 102. Movement of the mandrel distally within the longitudinal member 118 moves the anchors 102 distally through the lumen 120. As described above, the hook 104 and the projection 114 may be biased toward the hook shape and the laterally outward position, respectively, and constrained via an interior surface of the lumen 122 of the longitudinal member 118 when received therewithin. Thus, the anchors 102 are prevented (by frictional engagement with the wall of the lumen 120) from moving within the lumen 122 when the mandrel is not pushing the anchors 102 distally therethrough.

The distal jaw 124 is movably coupled to the distal end 120 via, for example, a connecting rod 126 received within a wall of the longitudinal member 118. Movement of the rod 126 moves the distal jaw 124 longitudinally relative to the longitudinal member 118. In particular, the distal jaw 124 is longitudinally movable relative to the longitudinal member 118 to increase and decrease the size of the space 130 between the distal end 120 of the longitudinal member 118 and the distal jaw 124, as desired. The distal jaw 124 may also include a slot 132 extending therethrough such that the anchor 102 may be passed longitudinally therethrough to be inserted into the target tissue 12. The slot 132 is also open to an edge 134 of the distal jaw 124 so that once the distal-most anchor 102 has been inserted into the target tissue 12, the anchor 102 may be slid laterally through the slot 132 to be deployed in the target tissue 12.

According to an exemplary method using the system 100, the delivery tool 108, including the anchors 102 housed within the lumen 122, is inserted into a body to target tissue therein via, for example, a working channel of an endoscope or other insertion device. The delivery tool 108 is then positioned over the target tissue 12 such that the target tissue 12 (e.g., tissue along a periphery of the tissue opening 10 to be closed) is received within the space 130 between the distal end 120 of the longitudinal element 118 and the distal jaw 124. Once the target tissue 12 has been received therein, the distal jaw 124 is drawn proximally toward the distal end 120 to hold the target tissue 12 therebetween. Holding the target tissue 12 between the distal end 120 and the distal jaw 124 provides a bracing force to press the tissue against to allow the anchor 102 to pierce through the held tissue.

The distal-most anchor 102 is then moved distally past the distal end 120 of the longitudinal member 118 for insertion into the target tissue 12. The distal tip 112 of the anchor 102 pierces the target tissue 12, extending distally past the distal surface 16 of the target tissue 12 and through the slot 132 of the distal jaw 124. Once the distal top 112 is inserted into the target tissue 12 to extend distally from the distal surface 16 thereof, the projection 114 is permitted to revert to its biased laterally outward position. In addition, as the anchor 102 is moved distally past the distal end 120 of the longitudinal member 118, the hook 104 is also permitted to revert to its biased configuration. Thus, once the anchor 102 has been inserted into the target tissue 12, the hook 104 extends proximally of the proximal surface 14 of the target tissue 12 while the projection 114 extends distally from the distal surface 16 to prevent the anchor 102 from being moved distally and/or proximally through the tissue once the anchor 102 has been placed.

Subsequent anchors 102 may be similarly placed in the tissue about a periphery of the tissue opening 10. Once the desired number of anchors 102 have been placed in a desired configuration about the tissue opening 10, a suture is looped about the periphery of the tissue opening engaging each of the hooks 104 of the placed anchors 102. The suture is then tightened, drawing all of the anchors 102, and thereby the tissue edges, toward one another, closing the tissue opening 10. As would be understood by those skilled in the art, a cincher may be slid over a portion of the suture to hold the suture in the tightened configuration. It will be understood by those of skill in the art, however, that the suture may be held in the tightened configuration via other methods. For example, the suture may be knotted.

In an alternative embodiment, rather than looping the suture about the anchors 102, the suture may be applied to the hooks 104 in a zig zag pattern. In particular, a distal end of the suture is tied or fixed to a first one of the placed anchors 102. A remaining length of the suture is then applied to anchors 102 in a zig zag pattern by moving the suture between hooks 104 on first and second opposing sides of the tissue opening 10 along a length thereof. Once the suture has been positioned to engage the hooks 104 in the zig zag pattern, the suture may be similarly tightened and fixed to close the tissue opening 10.

As shown in FIGS. 3-5, a system 200 according to another exemplary embodiment may be substantially similar to the system 100, comprising a plurality of anchors 202 to be placed in target tissue 22 about a periphery of a tissue opening 20 via a delivery tool 208. The delivery tool 208 may be substantially similar to the delivery tool 108.

The anchors 202 may also be substantially similar to the anchor 102. Rather than a projection, however, the anchors 202 may include a T-tag 214 at a distal end 210 thereof, as shown in FIG. 4. The T-tag 214 is biased toward a T-shaped configuration in which the T-tag extends transversely (e.g., perpendicularly) relative to a length of the anchor 202. In an insertion configuration, however, in which the T-tag is constrained by an interior surface of a lumen 122 of the delivery tool 108, the T-tag is moved to extend substantially parallel to the length of the anchor 202 to facilitate insertion thereof through the target tissue 22 from a proximal surface 24 of the target tissue 22. Once inserted through the target tissue 22 such that the T-tag 214 extends distally past a distal surface 26 of the target tissue 22, however, the T-tag 214 is permitted to revert to its biased T-shaped configuration to hold the anchor 202 in position within the target tissue 22.

In addition, rather than a single hook feature, each of the anchors 202 include a double head 204 at a proximal end 206 thereof which splits into two ends 204a, 204b that curve toward the distal end 210. Thus, the double head 204 includes two substantially hook-shaped ends 204a, 204b configured to receive a suture 250 therein, substantially as described above in regard to the system 100. Similarly to the anchors 102, multiple anchors 202 may be placed in tissue about a periphery of a tissue opening 20, as desired. Once the desired number of anchors 204 has been placed, a suture may be looped thereabout to engage one of the ends 204a, 204b of each anchor 202. The suture may then be tightened to close the tissue opening 20. Alternatively, rather than looping the suture about the tissue opening, the suture may be applied to the anchors 202 in a zig zag pattern, as described above in regard to the system 100.

In an alternative embodiment, as shown in FIG. 5, an anchor 202′ may include a split end 214′ at a distal end 210′ to act as the anchoring element. In particular, the split end 214′ may be substantially similar to the double head 204 described above in regard to the anchor 202. In addition to a double head 204′ at a proximal end 206′ however, the anchor 202′ includes two split ends 214a′, 214b′ at the distal end 210′, which curve toward the proximal end 206′. The split ends 214′ may be biased toward this curved configuration. In an insertion configuration, however, the two ends 214′, may be straightened and moved toward one another to facilitate piercing of the target tissue as it is inserted therethrough. Once the anchor 202′ has been inserted into the target tissue, however, the end 214′ is permitted to revert to the biased curved configuration to anchor the anchor 202′ therein.

As shown in FIGS. 6-7, a system 300 may be substantially similar to the systems 100, 200 described above, comprising a plurality of anchors 302 configured to engage tissue. The anchors 302 may be delivered to target tissue via a delivery tool 308 substantially similar to the deliver tools 108, 208 described above. The anchor 302 may be, for example, a tack extending from a proximal end 306 to a distal end 310. The proximal end 306 includes an opening 304 extending therethrough, the opening 304 sized and shaped to receive a suture 350 therethrough. A distal portion 314 of the anchor 302 is configured to engage target tissue into which it has been inserted. The distal portion 314 extends distally from the opening 304 to the distal end 310 and includes structures such as, for example, barbs, facilitating insertion into tissue while preventing the tack from disengaging the tissue once it has been inserted. Alternately, a surface of the distal portion 314 may be treated to provide a texture which facilitates engagement with the tissue. In another embodiment, the distal portion 314 may include a threading therealong for engaging tissue, the anchor 302 inserted into the target tissue via a torsional force.

The distal end 310 may include a recess 316 extending therein for engaging a proximal end of an immediately distal anchor 302. The recess 316 may be configured to receive the proximal end 306 in a non-rotative manner such that a rotation applied to a proximal-most one of the anchors 302 in the delivery tool 308 may translate to a distal-most one of the anchors 302.

The anchors 302 may be used in a manner substantially similar to the systems 100, 200. In particular, a desired number of anchors 302 may be inserted into target tissue surrounding a periphery of a tissue opening 30. Once the desired number of anchors 302 has been placed in target tissue, a suture 350 may be passed through the openings 304 in a loop or, alternatively, in a zig zag pattern and subsequently tightened to draw the anchors 302, and thereby tissue edges, toward one another to close the tissue opening 30. Although the suture 350 is described as being passed through the openings 304 after placement of the anchors 302 about the periphery of the tissue opening 30, it will be understood by those of skill in the art that the suture 350 may be pre-threaded through the openings 304, within the delivery tool 308.

As shown in FIGS. 8-9, a system 400 may be substantially similar to the systems 100-300 described above, comprising a plurality of anchors 402 to be inserted into a target tissue about a periphery of a tissue opening 40. The anchors 402 may be delivered to the target tissue via a delivery tool 408 that may be substantially similar to the delivery tools 108-308, described above. Similarly to the anchor 102, for example, each of the anchors 402 may include a sharpened distal tip 412 and a projection 414 for anchoring the anchor 402 in the target tissue. It will be understood by those of skill in the art, however, that the anchors 402 may include any of a variety of anchoring elements facilitating holding of the anchor 402 therein.

Rather than a hook at a proximal end 406 thereof for receiving a suture therein, however, each of the anchors 402 may include a tortuous path 404 formed in the proximal end 406 thereof for receiving a suture 450 therein. The tortuous path 404 may be formed of, for example, a hole 440 extending through a head portion 442 at the proximal end 406 of the anchor 402, along with a pair of hooks 444 extending proximally from the head portion 442. A space within each of the hooks 444 is configured to receive the suture therein, however, these holes are not aligned with one another such that a tortuous path is formed thereby.

The suture 450 is preassembled with the anchors 402. In particular, a distal end 452 of the suture 450 is connected to a distal-most one of the anchors 402a and a remaining length of the suture 450 is threaded through the holes 440 of each of the subsequent anchors 404 within the delivery tool 408. For example, the distal end 452 may be enlarged so that the distal end 452 is prevented from passing proximally through the hole 440 of the distal-most anchor 402. A remaining length is threaded proximally through each of the subsequent anchors 402.

According to an exemplary method, the distal-most anchor 402a is inserted into the tissue on a first side 44 of a tissue opening 40. Upon anchoring of the distal-most anchor 402a therein, a length of the suture 450 extending proximally therefrom is threaded through the pair of hooks 444 of the distal-most anchor 402a. The immediately proximal anchor 402b is then inserted into tissue on a second side 46 of the tissue opening 40 substantially opposing the first side 44. Upon anchoring of the immediately proximal anchor 402b, a length of the suture 450 extending proximally therefrom is threaded through the pair of hooks 444 of the immediately proximal anchor 402b. The suture 450 is then drawn proximally to tighten the suture 450, drawing the two anchors 402a, 402b toward one another. The tortuous paths of each of the anchors 402 prevents the tension of the suture 450 between each of the placed anchors 402 from becoming loosened. Subsequent anchors 402 may be similarly placed in a zig zag pattern, alternating between first and second sides 44, 46 of the tissue opening 40, along the length thereof.

As shown in FIGS. 10-11, a system 500 may be substantially similar to the system 400, described above, comprising a plurality of anchors 502, each of the anchors 502 including a tortuous path 504 through which a suture 550 may be threaded. Each of the anchors 502 may be substantially rod-shaped extending from a first end 506 to a second end 508 and including a first opening 540 and a second opening 544 extending laterally therethrough and in communication with one another such that the suture is threaded into the first opening 540 to exit out of the second opening 544. A distal end 552 of the suture 550 is fixed to a first one (e.g., distal-most) one of the anchors 502a. The anchors 502 may be delivered to the target tissue via a delivery tool substantially similar to the delivery tools described above. The anchors 502 may be longitudinally housed within the delivery tool with, for example, the first end 506 extending proximally and the second end 510 extending distally. Thus, the anchor 502 extends substantially parallel to portions of the suture 550 in the insertion configuration. Upon insertion into the target tissue, however, the anchor 502 moves laterally to extend substantially perpendicular to the suture 550, contacting a distal surface of the target tissue 52, as shown in FIG. 10. In use, the first anchor 502a is inserted into a first portion of tissue 54 on a first side of the tissue opening 50 and a second anchor 502b (e.g., an immediately proximal anchor 502) is inserted into a second portion of tissue 56 on a second side of the tissue opening 50. As described above in regard to the system 400, the suture 550 may be tightened to draw the two anchors 502a, 502b toward one another. The tortuous paths of the anchors 502 prevents a release of the tension of the suture 550 between the first and second anchors 502a, 502b. Subsequent anchors 502 are similarly placed about the tissue opening 50.

In an alternate embodiment, the first and second openings 540, 544 may not be in communication with one another. The suture 550 may be passed through the first opening 540 and then the second opening 544 to be releasably locked against an edge of the second opening 544. The suture 550 may, for example, be received within a recess or slot extending along the edge of the second opening 544 to be locked relative thereto.

As shown in FIGS. 12-14, a system 600 may be substantially similar to the systems 100-500, described above. Rather than anchors, however, the system 600 comprises a plurality of clips 602 delivered to target tissue within a patient's body via a delivery tool 608. The delivery tool 608 may be substantially tubular extending longitudinally from a proximal end (not shown) to a distal end 620 and including a lumen 622 extending therethrough. The clips 602 may be longitudinally stacked within the lumen 622 so that a mandrel 609 contacting a proximal-most one of the clips 602 may be moved distally relative to the delivery tool 608 to move a distal-most one of the clips 602 from the delivery tool 608 over a target tissue. Subsequent clips 602 are delivered to the target tissue about a periphery of a tissue opening 60. A suture 650 may be pre-assembled with the clips 602 so that, once the clips 602 have been positioned about the periphery, the suture 650 may be tightened to draw the clips 602 toward one another, closing the tissue opening 60.

Each of the clips 602 is movable between an open configuration, in which arms 614 thereof are separated from one another to receive target tissue therebetween, and a closed configuration, in which arms 614 are moved toward one another to grip the target tissue therebetween. The arms 614 may be formed of a shape memory material such that the arms 614 are biased in the closed configuration. When the clips 602 are received within the delivery tool 608, however, a proximal portion 606 of the clips 602 is constrained via an interior surface 623 of the lumen 622, holding the arms 614 in the open configuration. Thus, the clips 602 are held in the open configuration until the clips 602 are deployed (e.g., moved distally beyond the distal end 620) from the delivery tool 608.

As shown in FIG. 13, a distal-most one of the clips 602a is moved distally relative to the delivery tool 608 via, for example, the mandrel 609, such that only the arms 614 of the distal-most clip 602a extend distally past the distal end 620 of the delivery tool 608. Since the proximal portion 606 is still constrained via the interior surface 623 of the lumen 622, the arms 614 are held in the open configuration such that the target tissue may be received therebetween. Once the target tissue has been received therebetween, as desired, the distal-most clip 602a may be moved farther distally relative to the delivery tool 608, until the proximal portion 606 is no longer constrained via the interior surface 623 and the arms 614 are permitted to revert to their biased closed configuration, as shown in FIG. 14, gripping the tissue therebetween. Once the distal-most clip 602a has been deployed in the body, subsequent clips may be similarly placed along tissue about a periphery of the tissue opening 60.

The suture 650 may be preassembled with the clips 602. For example, the suture may be connected to the distal-most clip 602a and a remaining length threaded through an opening 604 extending through the proximal portion 606. Thus, upon placement of all the clips 602, the suture 650 may be tightened and cinched to close the tissue opening. Alternatively, rather than a suture, the system 600 may include an elastic band. The elastic band may be preassembled with the clips 602, substantially similarly to the suture 650, or may be stretched over the tissue opening 60 with the application of each of the subsequent clips 602.

Although the exemplary embodiment of the system 600 is described as including a mandrel 609 which contacts a proximal-most one of the clips 602 to move the clips 602 distally relative to the delivery tool 608, it will be understood by those of skill in the art that the system 600 may include alternative devices for moving the clips 602 relative to the delivery tool 608. For example, in an alternative embodiment, as shown in FIG. 15, clips 602′ may be moved relative to a delivery tool 608′ via a carrier member 609′ including features 607′ for engaging all of the clips 602′ received within a lumen 622′ of the delivery tool 608′. The carrier member 609′ is moved distally relative to the delivery tool 608′ to deploy clips 602′. Once a distal-most clip 602a′ is moved distally beyond a distal end 620′ of the delivery tool 608′ (e.g., a proximal portion 606′ is no longer constrained via an interior surface 623′ of the lumen 622′), the distal-most clip 602a′ becomes disengaged from the engaging feature 607′ of the carrier member 609′, deploying within the patient body.

As shown in FIG. 16, a system 700 according to another exemplary embodiment of the present disclosure may be substantially similar to the system 600 described above, comprising a plurality of clips 702 connected to one another via a suture 750 such that once the clips 702 have been placed along tissue about a periphery of a tissue opening, the suture 750 may be tightened to draw the clips 702 toward one another, thereby closing the tissue opening. Each of the clips 702 includes a plurality of arms 714 movable between an open tissue receiving configuration in which the arms 714 are separated from one another to receive tissue and a closed tissue gripping configuration in which the tissue received between the arms 714 are gripped thereby. It will be understood by those of skill in the art that the clips 702 may be configured in any of a variety of ways. In one exemplary embodiment, proximal ends of the arms 714 may be housed within a capsule 706 and movable relative thereto to move the clips 702 between the open and closed configurations. The arms 714 may be biased in the open configuration so that when the arms 714 extend distally out of a distal end of the capsule 706, the arms 714 are in the open tissue receiving configuration. When the target tissue has been received between the arms 714, the arms 714 may be moved proximally relative to the capsule 706 such that an interior surface of the capsule draws arms 714 toward one another to grip the tissue therebetween. In another exemplary embodiment, the arms 714 are biased toward the closed configuration, but held in the open tissue receiving configuration by a structure (e.g., an interior surface of a delivery tool substantially as described above in regard to the system 600) until each clip 702 is deployed into the target tissue.

The clips 702 may be coupled to one another via the suture 750. A distal end 752 of the suture 750 may be connected to a first one of the clips 702a and a remaining length thereof may extend through an opening 704 of each subsequent clip 702. The suture 750 may be threaded through the opening 704 of each subsequent clip 702 prior to loading of the clips in, for example, a delivery tool substantially similar to the delivery tool 608 described above in regard to the system 600. The suture 750 may also include ratcheting features 754 therealong such that the suture 750 may be drawn through the opening 704 in a proximal direction, while preventing the suture 750 from sliding back through the opening 704 in a distal direction. Thus, once the suture 750 has been tightened to draw clips 702 toward one another, the clips 702 are prevented from moving further apart from one another.

Similarly to the systems 100-600 described above, the clips 702 may be applied along a periphery of a tissue opening such that the suture 750 forms a loop thereabout. Alternatively, clips 702 may be placed alternatingly on opposing sides of the tissue opening such that the suture 750 forms a zig zag pattern thereacross. Once all of the clips 702 have been applied, as desired, the suture 750 may be tightened to draw the clips 702 toward one another, closing the tissue opening.

Although the delivery tool 108 has been specifically described and shown with respect to the system 100, it will be understood by those of skill in the art that any of the anchors/clips described above may be delivered to a target tissue using the delivery tool 108. In addition, the delivery tool 108 may be similarly used to deliver other tissue joining devices such as, for example, tissue spikes, staples, T-tags and pledgits, to target tissue to close a tissue opening or perforation thereof.

For example, as shown in FIG. 17, a system 800 may be substantially similar to the system 100, described above, comprising a delivery tool 808 through which a plurality of tissue joining devices 802 may be passed to treat, for example, a tissue opening or perforation. The delivery tool 808 is configured to be passed through, for example, a working channel of an endoscope, to a target tissue to be treated. The delivery tool 808 may be substantially similar to the delivery tool 108, including a longitudinal member 818 and a distal jaw 824 longitudinally movable relative thereto such that target tissue may be received within a space 830 between a distal end 820 of the longitudinal member 818 and the distal jaw 824. A first actuator of a handle assembly connected to the proximal end of the longitudinal member 818 may be used to move the distal jaw 824 longitudinally relative to the longitudinal member 818 between an open configuration, in which the distal jaw 824 and the distal end 820 of the longitudinal member 818 are separated from one another to receive the target tissue in the space 830 therebetween, and a closed configuration, in which the distal jaw 824 is drawn proximally toward the distal end 820 of the longitudinal member 818 to grip the target tissue therebetween. In particular, in the open configuration, opposing edges of a tissue opening or perforation may be drawn into the space 830 via, for example, a suction force. Once the target tissue is received in the space 830, the distal jaw 824 is drawn proximally toward the distal end 820 of the longitudinal member 818, gripping the target tissue to provide support thereto as it is being pierced by the tissue joining device 802.

The longitudinal member 818 extends from a proximal end (not shown) to the distal end 820 and includes a lumen 822 in which the tissue joining devices 802 may be movably housed. The distal jaw 824 may be movably connected to the longitudinal member 818 via a connecting portion 826 extending proximally from the distal jaw 824 and slidably received within a recess 829 extending longitudinally through a wall 828 of the longitudinal member 818. The connection portion 826 may be rigidly coupled to or integrally formed with the distal jaw 824. In one exemplary embodiment, the distal jaw 824 may extend substantially perpendicularly of the connection portion 826. The distal jaw 824, however, may extend at any acute angle relative to the connection portion 826 to facilitate tissue retention thereby.

A proximal surface 825 of the distal jaw 824 may include gripping features such as, for example, teeth, knurl, diamond point, or other patterns therealong or along an edge thereof to aid in the retention of tissue thereby. Although not shown, the distal jaw 824, similarly to the delivery tool 108, may also include a slot extending therethrough such that the tissue joining device 802 may be passed longitudinally therethrough to be inserted through the target tissue gripped by the distal jaw 824. A portion of the distal jaw 824 and/or a distal portion of the connecting portion 826 proximate the distal jaw 824 may include, for example, suction grates 860 through which a suction force may be applied to draw the target tissue into the space 830. The distal end 820 of the longitudinal member 818 may also include suction grates 862. Thus, the longitudinal member 818 and the connection portion 826 may form a vacuum channel extending therethrough to provide a suction force to the suction grates 860, 862. Suction passed through the suction grates 860, 862 draws the target tissue into the space 830 and holds the tissue therein as the delivery tool 808 is moved from the open configuration to the closed configuration. Although the delivery tool 808 is specifically shown and described as including suction grates 860, 862, the delivery tool 808 may include any of a variety of shapes, sizes and number of openings through which suction may be applied.

The tissue joining device 802 may be any of a variety of devices for closing a tissue opening such as, for example, tissue spikes, staples, T-tags and/or metallic or polymer loops. The tissue joining devices 802 may be movably housed within the lumen 822, each of the tissue joining devices 802 longitudinally aligned within the lumen 822. The tissue joining devices 802 may be moved distally relative to the longitudinal member 818 to pierce target tissue via a second actuator of the handle assembly. In another embodiment, the tissue joining device 802 may be a two-piece snap fit clip mechanism. In particular, a first piece of the of the snap clip may be movably housed within the lumen 822 of the longitudinal member 818 while a second piece of the snap clip may be housed or loaded in the distal jaw 824 so that when the first piece pierces the target tissue such that a distal end thereof extends distally past the target tissue, the distal end snaps into the corresponding second piece in the distal jaw 824. It will be understood by those of skill in the art that the multiple snap fit clip mechanisms may be loaded into the delivery tool 808.

The delivery tool 808 may, for example, be particularly suited to close tissue openings/perforations along a GI tract. According to an exemplary surgical technique using the system 800, the delivery tool 808 is inserted to a target site via a working channel of an endoscope. The delivery tool 808 may be inserted through the working channel in the open configuration. Alternatively, the delivery tool 808 may be inserted through the working channel in the closed configuration, and moved to the open configuration prior to positioning the delivery tool 808 relative to the tissue opening to be closed. Once the target site has been reached, the delivery took 808 is moved distally past the distal end of the endoscope such that, in the open configuration, the distal jaw 824 is positioned distally of the tissue opening and the distal end 820 of the longitudinal member 818 is positioned proximally of the tissue opening. Opposing edges of the tissue opening may be drawn into the space 820 between the distal end 820 and the distal jaw 824 via, for example, a suction force applied to the suction grates 860, 862. A suction force, however, is not required to be applied to both suction grates 860, 862. Suction may be applied to one of the grates 860, 862 or none of the grates 860, 862.

Upon receiving target tissue (e.g., opposing edges of the tissue opening) in the space 830, the delivery took 808 may be moved to the closed configuration by drawing the distal jaw 824 proximally relative to the longitudinal member 818 until the target tissue is gripped between the distal jaw 824 and the distal end 820 of the longitudinal member 818. The drawing of the distal jaw 824 and the distal end 820 toward one another approximates the opposing edges of the tissue opening, holding the tissue opening closed. The tissue joining devices 802 may then be moved distally relative to the longitudinal member 818 until a distal-most one of the tissue joining devices 802 is moved distally past the distal end 820 through the target tissue, joining the opposing edges of the tissue opening. This process may be repeated about the GI tract until the entire tissue opening has been closed using the tissue joining devices 802.

In another example, as shown in FIGS. 18 and 19, a system 900 is substantially similar to the system 800 described above, but is specifically designed to accommodate staples. A delivery tool 908 of the system 900 similarly comprises a longitudinal member 918 and a distal jaw 924 longitudinally movable relative thereto between an open configuration, in which the distal jaw 924 is separated from a distal end 920 of the longitudinal member 918 to permit target tissue to be received within a space 930 therebetween, and a closed configuration, in which the distal jaw 924 is drawn proximally toward the distal end 920 of the longitudinal member 918 to grip the target tissue therebetween. A lumen 922 extending through the longitudinal member 918, however, is specifically sized and shaped to accommodate a plurality of staples arranged longitudinally therein. In addition, a proximal surface 925 of the distal jaw 924 includes an anvil 932 for closing legs of the staples as they distally pierce the target tissue to come into contact with the anvil 930. Rather than suction grates, the delivery tool 908 is shown as including suction chambers 960, 962 along the distal jaw 924 and the distal end 920, respectively. The delivery tool 908, however, may include any shape, size and number of openings through which suction may be applied to draw tissue into the space 930.

In another example, as shown in FIGS. 20-24, a system 1000 is substantially similar to the systems 800 described above, but is specifically designed to accommodate a tissue spike 1002.

Similarly, a delivery tool 1008 of the system 1000 comprises a longitudinal member 1018 and distal jaw 1024 movable between an open configuration and a closed configuration. A lumen 1018 extending through the longitudinal member 1018, however, is specifically configured to slidably receive a plurality of tissue spikes 1002 in longitudinal alignment therein. In addition, the distal jaw 1024 includes an opening 1032 extending longitudinally therethrough to permit the spike 1002 to extend therethrough as it pierces a target tissue that is gripped between a distal end 1020 of the longitudinal member 1018 and the distal jaw 1024.

Each of the spikes 1002 may extend from a proximal end 1006 to a distal end 1010, the distal end 1010 including a sharpened distal tip 1012 for piercing the target tissue (e.g., opposing sides of a tissue opening) through which the spike 1002 is to be inserted. The distal end 1010 may also include projections 1014 movable between a tissue engaging configuration in which the projections 1014 extend laterally outward and an insertion configuration in which the projections are moved inward toward a longitudinal axis of the spike 1002 so that ends 1015 thereof are pointed toward the proximal end 1006. The projections may be biased in the laterally outward, tissue engaging position, but may be constrained in the insertion configuration. The projections 1014 may be constrained via an interior surface of the lumen 1022 or an engaging feature 1016 in the proximal end 1006 of an immediately distal spike 1002 in the lumen 1022. Thus, the projections 1014 are permitted to pass distally through the target tissue in the insertion configuration. Once the projections 1014 have been moved distally past the target tissue, however, the projections 1014 are permitted to revert to their biased, laterally outward position, to prevent the distal end 1010 from being drawn proximally through the target tissue.

The proximal end 1006 of each of the spikes 1002 includes an enlarged portion 1004, which has a cross-sectional area larger than a cross-sectional area of a distal portion of the spike 1002 such that the enlarged portion 1004 is prevented from passing distally through the target tissue. Thus, once the target tissue has been pierced by the distal tip 1012 so that the projections 1014 extend distally thereof, the target tissue is held between the projections 1014 and the enlarged portion 1004. As described above, the proximal end 1006 may also include an engaging feature 1016, in the form of a recess therein, for accommodating the distal end 1010 of an immediately proximal spike 1002 housed in the lumen 1022. Engagement between the engaging feature 1016 and the distal end 1010 of the immediately proximal spike 1002 permits a plurality of spikes 1002 to be held in longitudinal alignment within the lumen 1022 of the longitudinal member 1018.

The proximal end 1006 may also include a recess 1064 extending laterally therein for engaging a pin 1066 extending into the lumen 1022 at the distal end 1020 thereof. Engagement between the pin 1066 and the recess 1064 holds the spike 1002 relative to the longitudinal member 1018. As will be described in further detail below, a distal-most one of the spikes 1002 is held relative to the longitudinal member 1018 once the distal end 1010 of the distal-most one of the spikes 1002 has pierced the target tissue so that the delivery tool 1008 may be moved to the open configuration to disengage the distal jaw 1024 from the distal-most spike 1002, permitting the distal-most spike 1002 to be deployed in the target tissue.

The system 1000 may be used in a manner substantially similar to the system 800, described above. In particular, opposing sides of a tissue opening are received within the space 1030 between the distal jaw 1024 and the distal end 1020 of the longitudinal member 1018, when the delivery tool 1008 is in the open configuration. Once the target tissue (e.g., opposing sides of the tissue opening) has been received in the space 1030, the delivery tool 1008 may be moved to the closed configuration by drawing the distal jaw 1024 proximally toward the distal end 1020 of the longitudinal member 1018, thereby gripping the target tissue therebetween. The spike 1002 may be moved distally through the lumen 1022 until the distal-most one of the spikes 1002 pierces the target tissue and is moved distally therethrough. Since the target tissue is supported by the distal jaw 1024, piercing the target tissue causes the distal end 1010 of the distal-most spike 1002 to pass through the opening 1032 in the distal jaw 1024, as shown in FIG. 22, until the projections 1014 extend distally therepast, as shown in FIG. 23. As the distal-most one of the spikes 1002 pierces the target tissue, the pin 1066 at the distal end 1020 engages the recess 1064, preventing the distal-most one of the spikes 1002 from moving any farther distally relative to the longitudinal member 1018 and holding the distal-most one of the spikes 1002 relative thereto. While the distal-most spike 1002 is being held in the lumen 1022 via the pin 1066, the delivery tool 1008 is once again moved to the open configuration, to disengage the distal jaw 1024 from the distal end 1010 of the distal-most one of the spikes 1002, as shown in FIG. 24. Upon disengagement from the distal jaw 1024, the projections 1014 are permitted to revert to their biased, laterally outward position. The distal-most spike 1002 may then be deployed in the target tissue by pushing the plurality of spikes 1002 distally relative to the longitudinal member 1018 until the recess 1064 of the distal-most one of the spikes 1002 is disengaged from the pin 1066. Thus, opposing edges of the tissue opening are joined via the spike 1002 and held together between the enlarged portion 1004 and the laterally extending projections 1014. This process may be repeated until the entire tissue opening is closed, as desired.

It will be apparent to those skilled in the art that various modifications may be made in the present disclosure, without departing from the scope of the disclosure. Thus, it is intended that the present disclosure cover modifications and variations of this disclosure provided that they come within the scope of the appended claims and their equivalents.

1-15. (canceled)

16. A system for treating tissue, comprising:
a first anchoring device including a first anchoring element at a distal end thereof for anchoring the first anchoring device in a first target portion of tissue extending about a periphery of a tissue defect to be treated and a first suture receiving feature at a proximal end thereof;
a second anchoring device including a second anchoring element at a distal end thereof for anchoring the second anchoring device in a second target portion of tissue about the periphery of the tissue defect and a second suture receiving feature at a proximal end thereof; and
a suture configured to be passed through the first and second suture receiving features such that a tension applied to the suture draws the first and second anchoring devices toward one another to close the tissue defect.

17. The system of claim 16, further comprising:
a third anchoring device including a third anchoring element at a distal end thereof for anchoring the third anchoring device in a third target portion of tissue about the periphery of the tissue defect and a third suture receiving feature at a proximal end thereof.

18. The system of claim 16, wherein each of the first and second suture receiving features is a hook curving away from the proximal end of the first and second anchoring devices toward the distal ends thereof.

19. The system of claim 16, wherein each of the first and second anchoring elements is one of a barb, a projection, a threading and a T-tag.

20. The system of claim 16, further comprising:
a delivery tool including a longitudinal member extending longitudinally from a proximal end to a distal end and including a lumen extending therethrough, the lumen sized and shaped to movably receive the first and second anchoring devices longitudinally therein.

21. The system of claim 20, wherein the delivery tool further includes a distal jaw coupled to the longitudinal member such that the distal jaw is longitudinally movable relative thereto, a space between the distal end of the longitudinal member and the distal jaw configured to receive the target tissue therebetween so that one of the first and second anchoring devices may be passed therethrough.

22. The system of claim 21, wherein the distal jaw includes a slot extending longitudinally therethrough an open to a lateral edge thereof.

23. The system of claim 16, wherein each of the first and second receiving features includes a tortuous path.

24. The system of claim 23, wherein the tortuous path is formed of a hole extending through a proximal portion of each of the first and second anchoring devices and a pair of hooks extending proximally from the proximal portion, wherein a space extending through each of the pair of hooks is offset from one another.

25. The system of claim 23, wherein the tortuous path is formed of a first and second hole extending laterally through a portion of the first and second anchoring devices, the first and second holes in communication with one another.

26. A system for treating a tissue defect, comprising:
a delivery tube extending from a proximal end to a distal end and including a lumen extending therethrough;
a first clip movably housed within the lumen, the first clip including a first pair of arms at a distal end thereof, the pair of arms movable between an open configuration, in which the first pair of arms are separated from one another to receive a first portion of target tissue therebetween, and a closed configuration, in which the first pair of arms are drawn toward one another to grip tissue therebetween, the first clip biased toward the closed configuration so that when the first clip is received within the lumen of the delivery tube, an interior surface of the lumen constrains a proximal portion of the first clip to hold the first clip in the open configuration; and
a second clip movably housed within the lumen proximally of the first clip, the second clip including a second pair of arms at a distal end thereof, the second pair of arms movable between an open configuration and a closed configuration, the second clip biased toward the closed configuration so that when the second clip is received within the lumen of the delivery tube, an interior surface of the lumen constrains a proximal portion of the second clip to hold the second clip in the open configuration.

27. The system of claim 26, further comprising:
a suture connected to the first clip and extending through the proximal portion of the second clip such that, when the first and second clip have been applied to target portions along a periphery of a tissue defect, tensioning of the suture draws the first and second clip toward one another.

28. The system of claim 26, further comprising:
an elastic band configured to be gripped by the first and second pairs or arms.

29. The system of claim 27, wherein the suture includes ratcheting features along a portion of a length thereof, the ratcheting features permitting the suture to be drawn proximally through the proximal portion of the second clip while preventing the suture from sliding distally therethrough.

30. The system of claim 26, further comprising:
a mandrel configured to move the first and second clips distally relative to the delivery tube.

31. A method for treating a tissue defect, comprising:
placing a first anchoring device in a first target portion of tissue about a periphery of a tissue defect to be treated, the first gripping device including a first suture receiving feature at a proximal end thereof;
placing a second anchoring device in a second target portion of tissue about the periphery of the tissue defect, the second anchoring device including a second suture receiving feature at a proximal end thereof;
passing a suture through the first and second suture receiving features; and
tensioning the suture to draw the first and second anchoring devices toward one another.

32. The method of claim 31, further comprising:
anchoring a third anchoring device to a third target portion of tissue, the third anchoring device including a third suture receiving feature at a proximal end thereof.

33. The method of claim 31, wherein the first and second anchoring devices are anchored to the first and second target portions of tissue via one of a barb, a projection, a threading and a T-tag.

34. The method of claim 31, further comprising:
inserting a delivery tool to a target area within a patient body, a longitudinal member of the delivery tool housing the first and second anchoring devices; and
receiving the first target portions of tissue in a space defined between a distal end of a longitudinal member and a distal jaw coupled thereto; and
moving the distal jaw proximally relative to the longitudinal member to grip the target portion of tissue therebetween; and
moving the first anchoring device distally through the longitudinal member to be passed through the first target portion of tissue.

35. The method of claim 31, further comprising:
cinching the suture to maintain lock the first and second anchoring devices relative to one another.
